(19) United States
(12) Patent Application Publication
(10) Pub. No.: US 2021/0290529 A1
(43) Pub. Date: Sep. 23, 2021

(54) Edible High Nutrient Density Composite and the Use for Reduction and Management of Human Hypertension

(71) Applicant: Nutrient Foods, LLC, Reno, NV (US)

(72) Inventors: Charles Marsland, Reno, NV (US); Stacey Bell, Reno, NV (US)

(21) Appl. No.: 17/340,025

(22) Filed: Jun. 6, 2021

Related U.S. Application Data
(63) Continuation of application No. PCT/US2019/064967, filed on Dec. 6, 2019.
(60) Provisional application No. 62/776,118, filed on Dec. 6, 2018.

(51) Int. Cl. A61K 9/00; A61P 9/12; A61K 31/07; A61K 31/375; A61K 31/56; A61K 31/355; A61K 31/122; A61K 31/51; A61K 31/525; A61K 31/455; A61K 31/675; A61K 31/519; A61K 31/714; A61K 31/4178; A61K 31/19; A61K 31/14; A61K 33/42; A61K 33/18; A61K 33/06; A61K 33/30; A61K 33/34; A61K 33/32; A61K 33/04; A61K 33/24; A61K 33/00; A61K 33/26; A61K 31/198; A61K 31/4172; A61K 31/202; A23L 33/16; A23L 33/155; A23L 33/175; A23L 33/12; A23L 33/00; A23L 2/52; A23L 7/126; A23L 7/135; A23L 7/10

(52) U.S. Cl. CPC A61K 9/0056; A23V 2002/00; A61K 31/07; A61K 31/375; A61K 31/56; A61K 31/355; A61K 31/122; A61K 31/51; A61K 31/525; A61K 31/455; A61K 31/675; A61K 31/519; A61K 31/714; A61K 31/4178; A61K 31/19; A61K 31/14; A61K 33/42; A61K 33/18; A61K 33/06; A61K 33/30; A61K 33/34; A61K 33/32; A61K 33/04; A61K 33/24; A61K 33/00; A61K 33/26; A61K 31/198; A61K 31/4172; A61K 31/202; A61K 9/0095; A23L 33/16; A23L 33/155; A23L 33/175; A23L 33/12; A23L 33/40; A23L 2/52; A23L 7/126; A23L 7/135; A23L 7/101; A61P 9/12

(57) ABSTRACT

A method comprising an individual ingesting over a series of days a plurality of nutrient dense compositions, wherein the nutrient dense compositions comprise at least 5% (dry weight) of a nutrient formulation of vitamins, minerals, amino acids, fatty acids and fiber. In an example the use of the nutrient dense compositions is effective to achieve a blood pressure lowering of at least about 10%.

### BACKGROUND

This application relates to edible compositions with particular nutrient profiles.

The Centers for Disease Control and Prevention states that high blood pressure is a common and dangerous condition. Approximately 1 of 3 U.S. adults—or about 75 million people—have high blood pressure. Normal blood pressure is 120/80 and heart rate is 60-100 beats per minute (BPM). This common condition increases the risk for heart disease and stroke, which are two of the leading causes of death for Americans. Anti-hypertensive drugs are able to lower blood pressure between 10% and 20%. But this approach isn't working, because half of those prescribed drugs have not had their blood pressure normalized.

Lifestyle changes that include eating a low-sodium diet, exercising, and not smoking are the first recommended courses of treatment. Diet is the best non-pharmacologic way to help control high blood pressure. Most clinicians prescribe the DASH diet (Dietary Approaches to Stop Hypertension), but its impact on blood pressure lowering is minimal—about 7 mmHg for systolic and 4 mmHg for diastolic.

Sodium, the main dietary driver of hypertension, is over-consumed by all Americans starting at one year of age. Most dietary sodium comes from processed foods; canned foods; fast-foods; salty snacks; pizza; rice and noodle dishes; and restaurant foods. New thinking shows that potassium intake, represented by a potassium:sodium intake of greater than or equal to one, is more predictive of hypertension and cardiovascular disease (CVD) than either sodium or potassium intake alone. Only one-tenth of the population has a potassium:sodium greater than or equal to one. A low intake of omega-3 fatty acids (under the recommended amount of 1.6 grams/day for males and 1.1 g/day for females) also contributes to high blood pressure.

The diet that Americans are eating will never improve management of hypertension, let alone reverse it. It is high in sodium, devoid of potassium, and low in fruits, vegetables, whole grains, and low-fat dairy, which are all recommended in the DASH diet. New dietary strategies are needed.

### SUMMARY

This disclosure provides for a novel high nutrient density food composition the use of which is highly effective at lowering blood pressure in hypertensive subjects. An objective is to lower one or both of the systolic and diastolic blood pressures, preferably but not necessarily to levels beneath the commonly-accepted definitions of high blood pressure. The novel nutrient dense compositions are rich in essential vitamins, minerals, amino acids, omega-3 fatty acids, and fiber, while at the same time being low in sodium. Moreover, two previous studies showed these ingestible compositions induced satiety and produced significant weight loss. Weight loss of as little as 5% has been shown to produce reductions in both systolic and diastolic blood pressure readings. High nutrient density ingestible food compositions that target both blood pressure lowering and weight loss accomplish a novel, non-pharmacologic approach to the management of hypertension.

In one aspect, a novel composition and use for reducing the blood pressure of a human individual with hypertension comprises the individual ingesting over a series of days a plurality of nutrient dense compositions, wherein the nutrient dense compositions comprise at least 5% (dry weight) of a nutrient formulation of vitamins, minerals, amino acids, fatty acids and fiber, wherein the use of the nutrient dense compositions achieves at least the same blood pressure lowering effects as anti-hypertensive medications, of at least about 10%. The daily intake from the nutrient dense ingestible compositions may further comprise a potassium:sodium ratio of at least 1:1. The series of days may comprise at least 3 days. The formulation of nutrients may comprise each of the following nutrients: Vitamin A, Vitamin C, Vitamin D, Vitamin E, Vitamin K, Thiamin, Riboflavin, Niacin, Vitamin B6, Folate, Vitamin B12, Biotin, Pantothenic Acid, Choline, Phosphorus, Iodine, Magnesium, Zinc, Copper, Manganese, Selenium, Chromium, Molybdenum, Sodium, Potassium, Chloride, Calcium, Iron, Tryptophan, Threonine, Isoleucine, Leucine, Lysine, Methionine, Cystine, Phenylalanine, Tyrosine, Valine, Arginine, Histidine, Fiber, and Omega 3.

In an aspect, a method includes an individual ingesting over a series of days a plurality of nutrient dense compositions, wherein the nutrient dense compositions comprise at least 5% (dry weight) of a nutrient formulation of vitamins, minerals, amino acids, fatty acids and fiber.

The nutrient dense compositions may comprise at least 10% (dry weight) of the nutrient formulation. The nutrient dense compositions may comprise at least 25% (dry weight) of the nutrient formulation. The use of the nutrient dense compositions may also lower the weight of the user. The use of the use of the nutrient dense compositions may also increase the life expectancy of the consumer by reducing the risk of hypertension and cardiovascular disease.

The series of days may comprise at least eight weeks. The compositions may be selected from the group of compositions consisting of shakes, breakfast cereals, bars, and meals. The method may be effective to achieve a blood pressure lowering of at least about 10%.

In another aspect a method includes an individual ingesting over a series of at least three days a plurality of nutrient dense compositions, wherein the nutrient dense compositions comprise at least 10% (dry weight) of a nutrient formulation of vitamins, minerals, amino acids, fatty acids and fiber, wherein the nutrient dense compositions further comprise a potassium:sodium ratio of at least 1:1 and each of the following nutrients: Vitamin A, Vitamin C, Vitamin D, Vitamin E, Vitamin K, Thiamin, Riboflavin, Niacin, Vitamin B6, Folate, Vitamin B12, Biotin, Pantothenic Acid, Choline, Phosphorus, Iodine, Magnesium, Zinc, Copper, Manganese, Selenium, Chromium, Molybdenum, Sodium, Potassium, Chloride, Calcium, Iron, Tryptophan, Threonine, Isoleucine, Leucine, Lysine, Methionine, Cystine, Phenylalanine, Tyrosine, Valine, Arginine, Histidine, Fiber, and Omega 3.

### DETAILED DESCRIPTION

### Methods

### Subjects

Participants had confirmed hypertension by a healthcare professional.

### Dietary Intervention

For two months (i.e., at least 60 days) or in some case for at least eight weeks, each day the subjects consumed three nutrient dense compositions made to specification. Two consisted of one of a plurality of separate ingestible high nutrient compositions. One consisted of a high nutrient density food bar. Each contained a specific percentage of nutrients comprising vitamins, minerals, amino acids, fatty acids, and fiber. The nutrients in each of the nutrient dense edible compositions are as follows: Vitamin A, Vitamin C, Vitamin D, Vitamin E, Vitamin K, Thiamin, Riboflavin, Niacin, Vitamin B6, Folate, Vitamin B12, Biotin, Pantothenic Acid, Choline, Phosphorus, Iodine, Magnesium, Zinc, Copper, Manganese, Selenium, Chromium, Molybdenum, Sodium, Potassium, Chloride, Calcium, Iron, Tryptophan, Threonine, Isoleucine, Leucine, Lysine, Methionine, Cystine, Phenylalanine, Tyrosine, Valine, Arginine, Histidine, Fiber, and Omega 3.

Table A contains one non-limiting example of the amounts of each of these nutrients in an edible composition. Note that the percentages in Table A are exemplary of one edible composition and are not limiting of the scope of the present disclosure. To generalize the information in Table A, any composition herein can be designed to have a desired amount (in percent) of each nutrient.

TABLE A

| | Percent of Total (of nutrients |
|---|---|
| Nutrient | in the composition) |
| Vitamin A | 0.00384464% |
| Vitamin C | 0.15378549% |
| Vitamin D | 0.00002563% |
| Vitamin E | 0.06920347% |
| Vitamin K | 0.00020505% |
| Thiamin | 0.00384464% |
| Riboflavin | 0.00435726% |
| Niacin | 0.05126183% |
| Vitamin B6 | 0.00512618% |
| Folate | 0.00102524% |
| Vitamin B12 | 0.00001538% |
| Biotin | 0.00076893% |
| Pantothenic Acid | 0.02563091% |
| Choline | 1.40970030% |
| Phosphorus | 2.56309146% |
| Iodine | 0.00038446% |
| Magnesium | 1.02523658% |
| Zinc | 0.03844637% |
| Copper | 0.00512618% |
| Manganese | 0.00512618% |
| Selenium | 0.00017942% |
| Chromium | 0.00030757% |
| Molybdenum | 0.00019223% |
| Sodium | 0.97086798% |
| Potassium | 8.97082011% |
| Chloride | 2.33008315% |
| Calcium | 2.56309146% |
| Iron | 0.04613565% |
| Tryptophan | 0.77671924% |
| Threonine | 2.91269714% |
| Isoleucine | 3.88359618% |
| Leucine | 7.57301256% |
| Lysine | 5.82539427% |
| Methionine | 1.94179809% |
| Cystine | 0.77671924% |
| Phenylalanine | 2.42724761% |
| Tyrosine | 2.42724761% |
| Valine | 5.04867504% |
| Arginine | 1.28154573% |
| Histidine | 1.94179809% |
| Fiber | 38.83471911% |
| Omega 3 | 4.10094634% |
| Total | 100% |

The compositions used were all precision manufactured in a pharmaceutical grade facility utilizing advanced micro-dosing technology. All had a potassium:sodium of greater than one. All are quick preparation. The nutrient dense composition selection included: Shakes (chocolate and vanilla), Breakfast Cereal (maple almond, original, chocolate), Bars (honey granola, chocolate chip, peanut butter, chocolate), meals (Rice & beans, Pad Thai Noodles, Spicy Pad Thai Noodles, cheese & noodles, beef & noodles, chicken & noodles, cheese & rice, pad Thai rice, beef & rice, chicken & rice). Subjects were told to consume another meal on their own that was low sodium if possible.

Each of the edible high nutrient density compositions of this disclosure includes at least 5% (by dry weight) of the total nutrient composite formulation set forth above. More preferably, the amount of this nutrient composite formulation is at least 10%, and may be up to 20% or more. For example, a composition that weights 56 grams may have from 5 to 25 grams in total of the nutrients.

Following are illustrative non-limiting exemplary compositions of some of the edible high nutrient compositions.

Example composition of the high nutrient density Chocolate Chip Bar used:

| | | % of total |
|---|---|---|
| | Gram Weight | weight |
| Ferrous Bisglycinate Chelate | 0.01035970 | 0.02% |
| Copper Bisglycinate Chelate | 0.00481303 | 0.01% |
| Zinc Citrate | 0.01091336 | 0.02% |
| Potassium Gluconate | 0.89454847 | 1.30% |
| Tripotassium Citrate | 0.41290698 | 0.60% |
| Calcium Carbonate | 1.49851100 | 2.18% |
| Chromium Chloride | 0.00015881 | 0.00% |
| Magnesium Oxide | 0.33712439 | 0.49% |
| Manganese Sulfate | 0.00155648 | 0.00% |
| Potassium Iodide | 0.00004913 | 0.00% |
| Dipotassium Phosphate | 0.79421079 | 1.16% |
| L-Selenomethionine | 0.00361477 | 0.01% |
| Molybdenum | 0.00037180 | 0.00% |
| Vitamin A (Retinol Palmitate) | 0.00852221 | 0.01% |
| Vitamin B1 (Thiamin Mononitrate) | 0.00089646 | 0.00% |
| Vitamin B12 (Cyanocobalamin) | 0.00023285 | 0.00% |
| Vitamin B2 (Riboflavin) | 0.00072183 | 0.00% |
| Vitamin B3 (Niacinamide) | 0.00733469 | 0.01% |
| Vitamin B5 (D-calcium Pantothenate) | 0.00497129 | 0.01% |
| Vitamin B6 (Pyridoxine Hydrochloride) | 0.00089646 | 0.00% |
| D-Biotin | 0.00169979 | 0.00% |
| Folic Acid | 0.04860688 | 0.07% |
| Vitamin C (Ascorbic Acid) | 0.00158336 | 0.00% |
| Vitamin D3 (Cholecalciferol) | 0.02081655 | 0.03% |
| Vitamin E (DL Alpha-Tocopheryl Acetate) | 0.35554864 | 0.52% |
| Vitamin K1 (Phytonadione) | 0.00317837 | 0.00% |
| Maltodextrin | 0.00655465 | 0.01% |
| Choline | | |
| D-Biotin | 0.01040828 | 0.02% |
| Palm Kernel Oil | 0.06329668 | 0.09% |
| Oat Fiber | 0.05039977 | 0.07% |
| Whole Grain Rolled Oats | 6.64764143 | 9.68% |
| Crisp Rice | 5.59677690 | 8.15% |
| Soy Flake Concentrate | 9.06496559 | 13.19% |
| Milled Flax Seed | 1.34295786 | 1.95% |
| Milk Protein Isolate | 1.00721840 | 1.47% |
| Cinnamon | 0.16786973 | 0.24% |
| Natural Vanilla Type Powder | 0.09380000 | 0.14% |
| Sea Salt | 0.13429579 | 0.20% |
| Glycerin, Vegetable | 4.02887359 | 5.86% |
| Canola Oil | 1.67869733 | 2.44% |
| Semi-Sweet Chocolate Chips | 5.13500000 | 7.47% |
| Inulin Syrup | 4.86500000 | 7.08% |
| Tapioca Syrup | 17.83800000 | 25.96% |
| Natural Chocolate Flavor | 0.30000000 | 0.44% |
| Soy Flake Arcon | 6.25000000 | 9.10% |
| Total | 68.70590407 | 100.00% |

Example composition of the high nutrient density Beef Flavor Pack used:

| | Gram Weight | % of total |
|---|---|---|
| Ferrous Bisglycinate Chelate | 0.02045000 | 0.046670% |
| Copper Bisglycinate Chelate | 0.00660000 | 0.015062% |
| Zinc Citrate | 0.01581470 | 0.036092% |
| Potassium Gluconate | 0.97351695 | 2.221725% |
| Tripotassium Citrate | 0.44935736 | 1.025507% |
| Calcium Lactate | 2.59230165 | 5.916056% |
| Chromium Chloride | 0.00021143 | 0.000483% |
| Magnesium Oxide | 0.68750000 | 1.568987% |
| Manganese Sulfate | 0.00207221 | 0.004729% |
| Potassium Iodide | 0.00006541 | 0.000149% |
| Dipotassium Phosphate | 1.89437428 | 4.323272% |
| L-Selenomethionine | 0.00481250 | 0.010983% |
| Molybdenum | 0.00049500 | 0.001130% |
| Vitamin A (Retinol Palmitate) | 0.01134600 | 0.025893% |
| Vitamin B1 (Thiamin Mononitrate) | 0.00119350 | 0.002724% |
| Vitamin B12 (Cyanocobalamin) | 0.00031000 | 0.000707% |
| Vitamin B2 (Riboflavin) | 0.00096100 | 0.002193% |
| Vitamin B3 (Niacinamide) | 0.00976500 | 0.022285% |
| Vitamin B5 (D-calcium | 0.00661850 | 0.015104% |
| Pantothenate) | | |
| Vitamin B6 (Pyridoxine | 0.00119350 | 0.002724% |
| Hydrochloride) | | |
| D-Biotin | 0.01385700 | 0.031624% |
| Folic Acid | 0.00226300 | 0.005165% |
| Vitamin C (Ascorbic Acid) | 0.06471250 | 0.147684% |
| Vitamin D3 (Cholecalciferol) | 0.00210800 | 0.004811% |
| Vitamin E (DL Alpha-Tocopheryl | 0.02771400 | 0.063248% |
| Acetate) | | |
| Vitamin K1 (Phytonadione) | 0.00423150 | 0.009657% |
| Maltodextrin | 0.00872650 | 0.019915% |
| Choline Bitartrate | 0.45500125 | 1.038387% |
| Palm Kernel Oil | 0.08100177 | 0.184859% |
| Oat Fiber | 0.06449739 | 0.147194% |
| Pea Protein Isolate | 7.50000000 | 17.116226% |
| Soy Flake Concentrate | 7.50000000 | 17.116226% |
| Vegetable Blend | 5.00000000 | 11.410817% |
| Natural MiraPoix | 1.10000000 | 2.510380% |
| Roasted Beef Type Flavor | 2.75000000 | 6.275949% |
| Black Pepper | 0.30000000 | 0.684649% |
| Inulin | 6.00000000 | 13.692980% |
| Expeller Pressed Canola Oil | 6.00000000 | 13.692980% |
| Milled Flax Seed | 0.26500000 | 0.604773% |
| Total | 43.81807190 | 100% |

### Blood Pressure Monitor, and Standards for Blood Pressure and Heart Rate

All participants were provided the same blood pressure monitor, Medline MDS4001, Mundelein, Ill. 60060. The blood pressure readings are accurate to ±3 mmHg. Blood pressure and resting heart rate readings were obtained weekly at two times during the day—upon waking up in the morning and before bed.

Normal blood pressure is 120/80 and heart rate is 60-100 beats per minute (BPM).

### Quality of Life Questions

The participants completed the SF-12® quality of life questionnaire from Optum Insight Life Sciences, Inc., Johnston, R.I. This tool has been used in patients with hypertension. The SF-12 form was completed at the beginning, week 4, and week 8. Data were obtained at baseline, week 4, and week 8. Summary scores for physical health and mental health of the subjects are compared to the general population, matched for age and gender. In addition, estimated medical expenditures per month of the participants were compared to a healthy population.

Each week, participants rated a series of questions using a scale of one to five, with five being the best. Questions probed sleep, energy, mood, generally how they felt, passion, gastrointestinal function, fullness, appearance, and quality of foods consumed that were not provided to the participants.

### Classification of Weight and Waist Circumference

Classification of being overweight or obese using BMI kg/m² and normal or abnormal waist circumference was based on the National Heart, Lung, and Blood Institute of the National Institutes of Health. Being both overweight/obese and having a large waist circumference increases the risk of type 2 diabetes, hypertension, and heart disease. For males, normal waist circumference is less than or equal to 102 cm, and for females it is less than or equal to 88 cm.

### Study Design

The study lasted eight weeks and was prospective. Each participant was provided ingestible compositions as described above and the identical blood pressure monitoring device at no charge. Data collection forms were self-reported, completed weekly, and submitted electronically. At least weekly, the participants were offered online question and answer sessions.

### Statistics

All data are presented as means±standard deviations.

### Results and Discussion for all Subjects

All but one of the seven participants completed the 8-week study and most consumed the two nutrient-rich meals and one bar daily. One dropped out at week 6 due to gastroparesis. Baseline demographics are presented in Table 1. The average age was 56±4 years. All but one subject was with a mean BMI of 33±5 kg/m², and waist circumference means that exceeded normal for males and females. Each subject was hypertensive, with four having Stage 2 and three having either Elevated or Stage 1. Five of seven were taking medications to control blood pressure.

Blood pressure and resting heart rate data are presented in Table 2. Mean morning and evening blood pressure readings decreased between baseline and week 4, and again at week 8. At week 8, the mean morning systolic blood pressure was 125±13 mmHg and the mean diastolic blood pressure was 70±8 mmHg. Compared to baseline data, this represented a 21% decrease in systolic blood pressure and a 26% in diastolic pressure. Similarly, at week 8 for the evening blood pressure measurements, systolic pressure decreased 15% and diastolic decreased 19%. The comparison between week 8 and week 4 in the morning and evening showed that week 8 was 9% lower for both morning systolic and diastolic blood pressure, and 4% for both measurements in the evening. Resting heart rates did not change and were normal throughout the study.

The decrease in blood pressure observed over 8 weeks from consuming the ingestible compositions was between 15% and 26%. These findings are as good or better than anti-hypertensive drugs, which typically achieve a lowering effect of 10% to 20%. The edible high nutrient density compositions performed much better than the standard of care—the DASH diet. The DASH diet lowers systolic blood pressure by 7 mmHg and diastolic by 4 mmHg. In contrast, the regimen of high nutrient density composites lowered systolic blood pressure between 22 and 33 mmHg, and diastolic between 8 and 24 mmHg.

Mean body weight decreased 8 kg over 8 weeks, representing an 8% weight loss (Table 3). BMI units decreased 3 kg/m² over the 8 weeks. Males and females experienced reductions in waist circumferences: men by 3 cm at week 4, and women by 13 cm at week 8.

Quality of life using the SF-12 test and general questions improved over time (Table 4). For the SF-12 test, the summary scores for physical health and mental health of the subjects improved during the 8-week study. Physical health improved more than mental health compared to a healthy population. In addition, estimated medical expenditures of the participants decreased during the study. At the end, the cost per month decreased by $243 (56%).

Each quality of life indicator improved over the 8 week study, with the biggest improvements observed for a general feeling of wellbeing (65% increase) and in overall diet quality (60% increase) (Table 4). Energy level and passion increased by at least 50%.

In summary, a dietary intervention with nutrient dense composites reduced blood pressure equal to medications and much better than the DASH diet. Participants lost a significant amount of weight and found the diet was easy to follow. In addition, they felt better and estimated medical costs were predicted to decrease 55%.

### Individual Case Studies

### TM

TM is an obese 55-year-old African American woman, who is “on a mission to change her life”. She is happily married and has children and grandchildren. TM and her husband run a marketing business and her free time is devoted to her church; she was appointed to Executive Secretary for the bishop. Her blood pressure has been poorly controlled despite being on blood pressure lowering medications for 30 years. At the start of the study she was taking Carvedilol, a beta-blocker, 25 mg b.i.d., and the same drug at 6.25 mg once a day.

After four weeks, TM's blood pressure was lower than her baseline values, and she lost 6 kg and 9 cm from her waist. At week 8, her diastolic blood pressure normalized, but her systolic was still elevated. TM remained weight stable and lost another centimeter from her waist circumference. These findings are remarkable because during the 8-week study, TM developed pneumonia and had to be hospitalized, and underwent orthoscopic knee surgery.

| Time | Baseline | Week 4 | Week 8 |
|---|---|---|---|
| Morning | | | |
| Blood pressure (mm | 209/124 | 128/85 | 135/73 |
| Hg) | | | |
| Heart rate (BPM*) | 70 | 55 | 64 |
| Evening | | | |
| Blood pressure (mm | 154/98 | 131/81 | 137/80 |
| Hg) | | | |
| Heart rate (BPM) | 56 | 63 | 66 |

*Beats per minute

### ME

ME is a 55-year-old female, who had a hemorrhagic stroke nine years ago causing her to be mostly in a wheelchair and homebound. Before she was a manager in a large retail store. She has been married for 16 years and has one daughter and lots of pets. She is highly motivated to reduce her blood pressure, yet realizes that she'll need to remain on medication for life. She stated that she has more energy after starting the program, and even attempted rock climbing.

After four weeks, her blood pressure normalized, while continuing with 40 mg Lisinopril, an ACE inhibitor, and a diuretic (20 mg, yet not specified). Her body weight was stable, yet she was normal weight upon entry into the study; her waist circumference decreased 5 cm at week 4. At week 8, her blood pressure continued to be normal and she did not lose more weight or inches.

| Time | Baseline | Week 4 | Week 8 |
|---|---|---|---|
| Morning | | | |
| Blood pressure (mm | 125/85 | 110/69 | 101/69 |
| Hg) | | | |
| Heart rate (BPM*) | 98 | 70 | 68 |
| Evening | | | |
| Blood pressure (mm | 124/84 | 113/70 | 105/71 |
| Hg) | | | |
| Heart rate (BPM) | 89 | 75 | 71 |

*Beats per minute

### GL

GL is a 62-year-old single, unemployed male with one child. In early 2018, he started driving for Lyft and gained weight due to eating poorly. He has always been athletic, and particularly enjoys boxing. He thought he was in excellent health until he saw a dentist for routine care, and was identified as having Stage 1-2 high blood pressure. Instead of starting on medications to control his blood pressure, he enrolled in this study. He was also classified as Obese, based on his BMI of 30 kg/m², and had a waist circumference approaching an unhealthy amount (99 cm).

After 4 weeks, his blood pressure was normal. In addition, he lost 5 kg and 8 cm from his waist circumference. He is now considered to be Overweight, rather than Obese with a BMI of 28.5 kg/m². He remarked how lean he felt at the time. At week 8, his blood pressure remained normal, although it was trending upward. He lost another 1 kg and his waist circumference did not change.

| Time | Baseline | Week 4 | Week 8 |
|---|---|---|---|
| Morning | | | |
| Blood pressure (mm | 152/98 | 118/76 | 128/78 |
| Hg) | | | |
| Heart rate (BPM*) | 53 | 56 | 56 |
| Evening | | | |
| Blood pressure (mm | 131/87 | 116/67 | 130/80 |
| Hg) | | | |
| Heart rate (BPM) | 71 | 59 | 58 |

*Beats per minute

### CW

This 50-year-old African American woman has a lot to live for; she has two sons in their 20s and hopes that she'll be a grandmother soon. Yet, she has a strong family history of CVD, type 2 diabetes, and hypertension. Her blood pressure at the beginning of the study was Stage 1 hypertension, and she was Obese with a BMI of 35 kg/m². Daily she takes 10 mg amlodipine, a calcium channel blocker, losartan, 50 mg, which is an angiotensin receptor blocker (ARB) and an unspecified diuretic.

At week four, her blood pressure was lower than baseline, but still elevated, especially at night. However, her physician reduced her amlodipine in half and eliminated the diuretic. She lost 3 kg and 10 cm from her waist circumference. At week 8, her blood pressure normalized, weight was stable, and her waist circumference went down another 10 cm.

| Time | Baseline | Week 4 | Week 8 |
|---|---|---|---|
| Morning | | | |
| Blood pressure (mm | 135/86 | 118/70 | 119/77 |
| Hg) | | | |
| Heart rate (BPM*) | 79 | 81 | 71 |
| Evening | | | |
| Blood pressure (mm | 132/80 | 129/85 | 126/81 |
| Hg) | | | |
| Heart rate (BPM) | 97 | 83 | 75 |

*Beats per minute

### AB

AB is a 57-year-old African American woman with Stage 2 hypertension, hypercholesterolemia, and type 2 diabetes. She was Obese based on her BMI of 37 kg/m² and had an unhealthy waist circumference of 112 cm. Her pride is her three grown children and five grandchildren. She hopes to reduce some of her blood pressure medication use. Currently she is taking (an unspecified diuretic) 25 mg once a day; amlodipine (calcium channel blocker) 10 mg once a day; and metoprolol tartrate (beta blocker) 100 mg b.i.d.

After four weeks, her blood pressure had not changed and she gained 1 kg, but she lost 5 cm from her abdomen. She states that she is full of energy and enjoying the program. At week 8, her blood pressure normalized; she lost 6 kg and her waist circumference did not change.

| Time | Baseline | Week 4 | Week 8 |
|---|---|---|---|
| Morning | | | |
| Blood pressure (mm | 178/90 | 144/83 | 129/69 |
| Hg) | | | |
| Heart rate (BPM*) | 67 | 84 | 68 |
| Evening | | | |
| Blood pressure (mm | 140/82 | 137/72 | 123/67 |
| Hg) | | | |
| Heart rate (BPM) | 60 | 91 | 64 |

*Beats per minute

### RS

RS is a busy, 52-year-old grandmother, who manages a delicatessen and has a busy family life. She is Obese, according to her BMI (34 kg/m²) and has an unhealthy waist circumference, indicating a very high risk for high blood pressure, type 2 diabetes, and heart disease. In addition to hypertension (Stage 2), she also has type 2 diabetes and hypercholesterolemia, but is not taking medications for any of these.

At week 4, she normalized her diastolic blood pressure (between 60 and 70 mm Hg) and lost 3 kg and 14 cm from her abdomen. By week 8, her diastolic blood pressure remained normal but her systolic was still elevated. She lost another 6 kg and reduced her waist circumference another 9 cm.

| Time | Baseline | Week 4 | Week 8 |
|---|---|---|---|
| Morning | | | |
| Blood pressure (mm | 153/84 | 158/72 | 135/55 |
| Hg) | | | |
| Heart rate (BPM*) | 74 | 67 | 65 |
| Evening | | | |
| Blood pressure (mm | 184/92 | 135/63 | 140/55 |
| Hg) | | | |
| Heart rate (BPM) | 63 | 69 | 67 |

*Beats per minute

### MH

Mr. MH is a 60-year-old with Stage 2 hypertension, obesity (BMI 34 kg/m²), and a large waist circumference placing him at high risk for hypertension, heart disease, and type 2 diabetes. He is managed with a beta blocker (Labetalol), losartan (ARB), and clonidine (a centrally-acting alpha-agonist). In addition, he has type 2 diabetes, for which he uses insulin, and has neuropathy and hypercholesterolemia. MH was not always able to consume the recommended amounts of the nutrient-rich meals and bars due to epi-gastric pain. He had no change in blood pressure at week 4, but did experience a 6-kg weight loss with no change in waist circumference. At week 6, he developed diabetic gastric paresis and withdrew from the study at his physician's advice.

| Time | Baseline | Week 4 | Week 8 |
|---|---|---|---|
| Morning | | | Withdrew from study |
| Blood pressure (mm | 157/90 | 174/86 | |
| Hg) | | | |
| Heart rate (BPM*) | 80 | 70 | |
| Evening | | | |
| Blood pressure (mm | 180/90 | 160/86 | |
| Hg) | | | |
| Heart rate (BPM) | 80 | 70 | |

*Beats per minute

TABLE 1
Baseline demographic information

| | Number or Mean ± standard deviation |
|---|---|
| Attribute | (S.D.) |
| Gender | 62% female |
| Age (years) | 56 ± 4 |
| Body Mass Index | 33 ± 5 |
| (kg/m²) | |
| Classification of BMI | Normal = 1 |
| | Overweight = 0 |
| | Obese = 6 |
| Waist circumference | Males: 109 ± 14 cm (high risk > 102 cm) |
| (cm) | Females: 102 ± 12 cm (high risk > 88 cm) |
| Co-morbidities | 5/7 have metabolic disorders (e.g., diabetes, |
| | hypercholesterolemia) |
| | 1/7 has none |
| | 1/7 has joint and muscular skeletal pain |
| Blood pressure | 5/7 |
| lowering medication use | |

TABLE 2
Blood pressure and resting heart rate changes over two months

| Time | Baseline | Week 4 | Week 8 |
|---|---|---|---|
| Morning | | | |
| Blood pressure | 158 ± 28/94 ± | 136 ± 24/77 ± | 125 ± 13/70 ± 8* |
| (mm Hg) | 13 | 7 | |
| Heart rate (BPM+) | 74 ± 14 | 69 ± 11 | 65 ± 5 |
| Evening | | | |
| Blood pressure | 149 ± 24/88 ± | 132 ± 16/75 ± | 127 ± 13/72 ± 10 |
| (mm Hg) | 6 | 9 | |
| Heart rate (BPM+) | 74 ± 16 | 73 ± 11 | 67 ± 6 |

*Compared to baseline, this was a 21% decrease in systolic blood pressure and a 26% decrease in diastolic. Compared to week 4, this was a 9% decrease for both systolic and diastolic blood pressure.
+Beats per minute
\hat{ }Compared to baseline, this was a 15% decrease in systolic blood pressure and a 19% decrease in diastolic. Compared to week 4, this was a 4% decrease for both systolic and diastolic blood pressure.

TABLE 3
Change in body weight, waist circumference, and body mass index

| | Baseline | Week 4 | Week 8 |
|---|---|---|---|
| Body weight (kg) | 96 ± 19 | 93 ± 17 | 88 ± 17 |
| Waist circumference | 109 ± 14 | 106 ± 21 | 91 |
| (cm): males | | | |
| Waist circumference | 102 ± 12 | 93 ± 11 | 89 ± 12 |
| (cm): females | | | |
| Body mass index | 33 ± 5 | 32 ± 5 | 30 ± 8 |
| (kg/m²) | | | |

TABLE 4
Changes in quality of life and general feeling of wellbeing

| Time | Baseline | Week 4 | Week 8 |
|---|---|---|---|
| SF-12 test | | | |
| Physical component | −43% above | −71% above | −100% above |
| summary compared to | −43% below | −14% below | |
| a healthy population | | | |
| Mental component | −29% above | −29% above | −67% above |
| summary compared to | −43% below | −43% below | −17% below |
| a healthy population | | | |
| Cost of care compared | 219% higher ($437 | 129% higher ($314 | 42% higher ($194 vs. |
| to a healthy | vs. $137 | vs. $137) | $137) |
| population per month | | | |
| Other measurements* | | | |
| General feeling of | 2.6 ± 1 | 3.8 ± 1.2 | 4.3 ± 0.5 |
| wellbeing | | | |
| Fullness | 3.0 ± 0.6 | 3.6 ± 0.8 | 4.2 ± 0.4 |
| Moodiness | 3.4 ± 1.3 | 3.8 ± 1.2 | 4.0 ± 1.7 |
| Energy | 3.1 ± 1.1 | 4.0 ± 1.1 | 4.7 ± 0.5 |
| Gastrointestinal | 3.3 ± 1.4 | 3.9 ± 1.2 | 4.5 ± 0.8 |
| symptoms | | | |
| Sleep | 2.7 ± 1 | 3.9 ± 1.1 | 4.0 ± 0.9 |
| Appearance (e.g., | 2.9 ± 0.7 | 3.6 ± 0.8 | 4.2 ± 0.4 |
| hair, skin, nails) | | | |
| Diet quality | 3.0 ± 0.6 | 4.0 ± 1.2 | 4.8 ± 0.4 |
| Passion | 1.7 ± 1.1 | 3.3 ± 1.7 | 4.3 ± 1.2 |

*The scale used was 1 to 5, with 5 indicating the best state to be in (e.g., best mood, no gastrointestinal symptoms).

A number of implementations have been described. Nevertheless, it will be understood that additional modifications may be made without departing from the scope of the inventive concepts described herein, and, accordingly, other examples are within the scope of the following claims.

What is claimed is:

1. A method, comprising:
an individual ingesting over a series of days a plurality of nutrient dense compositions, wherein the nutrient dense compositions comprise at least 5% (dry weight) of a nutrient formulation of vitamins, minerals, amino acids, fatty acids and fiber.

2. The method of claim 1, wherein the daily intake from the nutrient dense ingestible compositions further comprises a potassium:sodium ratio of at least 1:1.

3. The method of claim 1, wherein the series of days comprises at least three days.

4. The method of claim 1 wherein the formulation of nutrients comprises each of the following nutrients: Vitamin A, Vitamin C, Vitamin D, Vitamin E, Vitamin K, Thiamin, Riboflavin, Niacin, Vitamin B6, Folate, Vitamin B12, Biotin, Pantothenic Acid, Choline, Phosphorus, Iodine, Magnesium, Zinc, Copper, Manganese, Selenium, Chromium, Molybdenum, Sodium, Potassium, Chloride, Calcium, Iron, Tryptophan, Threonine, Isoleucine, Leucine, Lysine, Methionine, Cystine, Phenylalanine, Tyrosine, Valine, Arginine, Histidine, Fiber, and Omega 3.

5. The method of claim 4, wherein the nutrient dense compositions comprise at least 10% (dry weight) of the nutrient formulation.

6. The method of claim 4, wherein the nutrient dense compositions comprise at least 25% (dry weight) of the nutrient formulation.

7. The method of claim 1, wherein the use of the nutrient dense compositions lowers the weight of the individual.

8. The method of claim 1, wherein the use of the use of the nutrient dense compositions increases the life expectancy of the individual by reducing the risk of hypertension and cardiovascular disease.

9. The method of claim 1, wherein the series of days comprises at least eight weeks.

10. The method of claim 1, wherein the compositions are selected from the group of compositions consisting of shakes, breakfast cereals, bars, and meals.

11. The method of claim 1, wherein the method is effective to achieve a blood pressure lowering of at least about 10%.

12. A method, comprising:
an individual ingesting over a series of at least three days a plurality of nutrient dense compositions, wherein the nutrient dense compositions comprise at least 10% (dry weight) of a nutrient formulation of vitamins, minerals, amino acids, fatty acids and fiber, wherein the nutrient dense compositions further comprise a potassium:sodium ratio of at least 1:1 and each of the following nutrients: Vitamin A, Vitamin C, Vitamin D, Vitamin E, Vitamin K, Thiamin, Riboflavin, Niacin, Vitamin B6, Folate, Vitamin B12, Biotin, Pantothenic Acid, Choline, Phosphorus, Iodine, Magnesium, Zinc, Copper, Manganese, Selenium, Chromium, Molybdenum, Sodium, Potassium, Chloride, Calcium, Iron, Tryptophan, Threonine, Isoleucine, Leucine, Lysine, Methionine, Cystine, Phenylalanine, Tyrosine, Valine, Arginine, Histidine, Fiber, and Omega 3.
